(19) United States
(12) Patent Application Publication
(10) Pub. No.: US 2023/0203474 A1
(43) Pub. Date: Jun. 29, 2023

(54) COMPOUND, SOLID CARRIER INCLUDING THE SAME AND METHOD FOR PREPARING NUCLEIC ACID

(71) Applicant: Industrial Technology Research Institute, Hsinchu (TW)

(72) Inventors: Ming-Chun Lin, Hsinchu City (TW); Hui-Ling Cheng, Hsinchu City (TW); Chun-Ting Lai, Taoyuan City (TW); Hua-Cheng Chou, Taichung City (TW); Wei-Chin Huang, Tainan City (TW); Chih-Hung Chen, Tainan City (TW); Shu-Feng Chen, Changhua County (TW)

(73) Assignee: Industrial Technology Research Institute, Hsinchu (TW)

(21) Appl. No.: 18/088,628

(22) Filed: Dec. 26, 2022

Related U.S. Application Data
(60) Provisional application No. 63/294,057, filed on Dec. 27, 2021.

(30) Foreign Application Priority Data
Nov. 17, 2022 (TW) 111143952

(51) Int. Cl. C12N 15/10; C07H 21/00; C07D 491/18; C07H 1/06

(52) U.S. Cl. CPC C12N 15/1006; C07D 491/18; C07H 1/06; C07H 21/00

(57) ABSTRACT

A compound, a solid carrier including the same and a method for preparing a nucleic acid are provided. The compound has a structure represented by Formula (1) as follows.

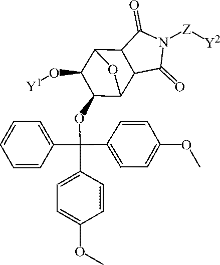

In Formula (1), the definition of Y¹, Y², Z and * are the same as defined in the detailed description.

### CROSS REFERENCE TO RELATED APPLICATION

This application claims the priority benefit of U.S. provisional application serial no. 63/294,057, filed on Dec. 27, 2021, and Taiwan application serial no. 111143952, filed on Nov. 17, 2022. The entirety of the above-mentioned patent application is hereby incorporated by reference herein and made a part of specification.

### TECHNICAL FIELD

The technical field relates to a compound, a solid carrier including the same and method for preparing a nucleic acid.

### BACKGROUND

With the vigorous development of biotechnology, nucleic acids are used in large quantities. For this reason, the demand for preparing nucleic acids has gradually increased. However, when the currently commercially available column for preparing nucleic acid cuts the bond between the nucleic acid and the solid carrier, the solid carrier will degrade, so that the solid carrier can only be used once. Thereby, the cost of preparing nucleic acid is increased.

### SUMMARY

One of exemplary embodiments comprises a compound having a structure represented by Formula (1) as follows.

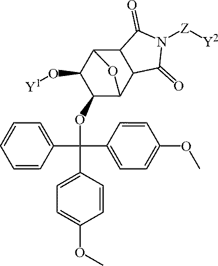

In Formula (1), Y¹ represents hydrogen or acetyl group;
Y² represents hydrogen, *—COOH, *—NH₂ or *—NCO;
Z represents a divalent organic group, and
* represents a bonding position.

One of exemplary embodiments comprises a solid carrier including the aforementioned compound.

One of exemplary embodiments comprises a method for preparing a nucleic acid including the aforementioned solid carrier.

Several exemplary embodiments are described in detail below to further describe the disclosure in details.

### DETAILED DESCRIPTION OF DISCLOSED EMBODIMENTS

Hereinafter, embodiments of the disclosure are described in detail. The details provided in the embodiments are exemplary, and are not intended to limit the scope of the disclosure. Those having ordinary skill in the art may modify or change the details according to the requirements of actual implementation.

The “divalent organic group” as used in the specification is a substituent derived from an organic compound, which is derived from a group formed by removing two hydrogen atoms from an atom of an organic compound. Thereby, two chemical bonds may be formed with other atoms.

One of exemplary embodiments comprises a compound having a structure represented by Formula (1) as follows. The structure represented by Formula (1) will be described in detail.

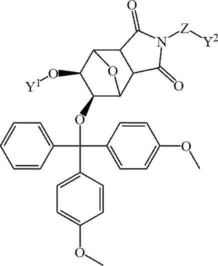

In Formula (1), Y¹ represents hydrogen or acetyl group;
Y² represents hydrogen, *—COOH, *—NH₂ or *—NCO;
Z represents a divalent organic group, and
* represents a bonding position.

In this embodiment, Z may include alkylene group, *—COO—*, arylene group, *—HNCO—*, *—NHCONH—*, heterocycloalkylene group, heteroarylene group or a combination thereof, preferably alkylene group, *—COO—*, *—HNCO—* or a combination thereof, more preferably alkylene group, *—COO—* or a combination thereof; * represents a bonding position.

In this embodiment, the structure represented by Formula (1) includes a structure represented by Formula (2) as follows.

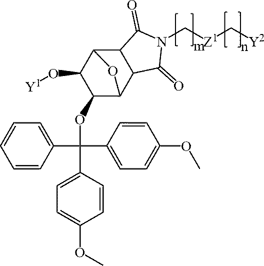

In Formula (2), Y¹ represents hydrogen or acetyl group;
Y² represents hydrogen, *—COOH, *—NH₂ or *—NCO;
Z¹ represents a divalent organic group;
m represents an integer from 0 to 8;
n represents an integer from 0 to 6, and
* represents a bonding position.

In this embodiment, Z¹ may include *—COO—*, arylene group, *—HNCO—*, *—NHCONH—*, heterocycloalkylene group, heteroarylene group or a combination thereof, preferably *—COO—* or *—HNCO—*, more preferably *—COO—*; m is preferably an integer from 0 to 5; n is preferably an integer from 0 to 6; * represents a bonding position.

The compound represented by Formula (1) may be selected from one of Compound 1 represented by Formula (1-1) as follows, Compound 2 represented by Formula (1-2) as follows, Compound 4 represented by Formula (1-3) as follows, Compound 5 represented by Formula (1-4) as follows and Compound 6 represented by Formula (1-5) as follows.

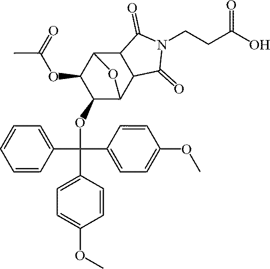 [SMILES: COc1ccc(C(O[C@@H]2C3OC(C4C(=O)N(CCC(=O)O)C(=O)C43)[C@@H]2OC(C)=O)(c2ccccc2)c2ccc(OC)cc2)cc1]

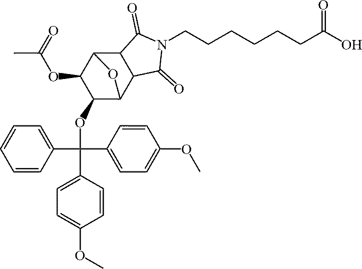 [SMILES: COc1ccc(C(O[C@@H]2C3OC(C4C(=O)N(CCCCCCC(=O)O)C(=O)C43)[C@@H]2OC(C)=O)(c2ccccc2)c2ccc(OC)cc2)cc1]

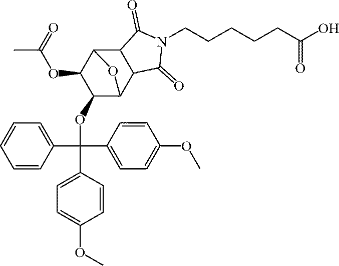 [SMILES: COc1ccc(C(O[C@@H]2C3OC(C4C(=O)N(CCCCCC(=O)O)C(=O)C43)[C@@H]2OC(C)=O)(c2ccccc2)c2ccc(OC)cc2)cc1]

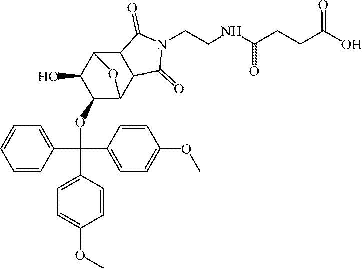 [SMILES: COc1ccc(C(O[C@@H]2C3OC(C4C(=O)N(CCNC(=O)CCC(=O)O)C(=O)C43)[C@@H]2O)(c2ccccc2)c2ccc(OC)cc2)cc1]

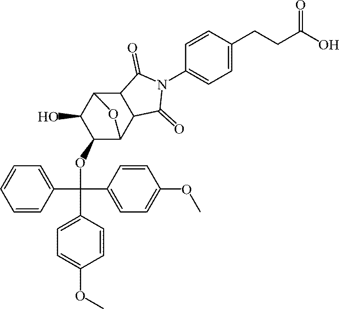 [SMILES: COc1ccc(C(O[C@@H]2C3OC(C4C(=O)N(c5ccc(CCC(=O)O)cc5)C(=O)C43)[C@@H]2O)(c2ccccc2)c2ccc(OC)cc2)cc1]

One of exemplary embodiments comprises a solid carrier including the aforementioned compound. The solid carrier may further include a substrate whose surface has a linking molecule. The compound may bond to the linking molecule on the surface of the substrate to immobilize the compound on the substrate. Thereby, the solid carrier to which the above compound is bonded may be used for a preparation of a nucleic acid. The substrate is not particularly limited, for example, a stainless-steel substrate or other suitable substrates may be selected.

In this embodiment, a terminal functional group of the linking molecule may include amino group or carboxyl group. For example, the linking molecule may include aminosilane-based compound (e.g. aminopropyltriethoxysilane) or other suitable compounds. The terminal functional group of the linking molecule bonds to the aforementioned compound to immobilize the compound on the substrate. An amide bond or a urea functional group may be formed between the terminal functional group of the linking molecule and the compound. For example, when the compound has a structure represented by Formula (1) with Y² representing *—COOH, and the terminal group of the linking molecule is amino group, the amide bond may be formed between the compound and the linking molecule. When the compound has a structure represented by Formula (1) with Y² representing *—NH₂, and the terminal group of the linking molecule is carboxyl group, the amide bond may be formed between the compound and the linking molecule. When the compound has a structure represented by Formula (1) with Y² representing *—NCO, and the terminal group of the linking molecule is amino group, the urea functional group may be formed between the compound and the linking molecule.

The synthesis method and conditions of bonding the compound to the linking molecule on the surface of the substrate are not particularly limited. For example, known organic synthesis methods may be used.

One of exemplary embodiments comprises a method for preparing a nucleic acid including the aforementioned solid carrier. The method for preparing a nucleic acid is not particularly limited. For example, known methods for preparing a nucleic acid may be used. For example, in the method of using the solid carrier for preparing a nucleic acid, a nucleic acid precursor may be used to replace dimethoxytriphenylmethyl (4,4′-dimethoxytriphenylmethyl, DMT) in the compound during the process of synthesizing the nucleic acid sequence; next, degradation is performed with ammonia water to fall off the nucleic acid precursor and obtain a prepared nucleic acid and a solid carrier to which degraded compound is bonded. During the degradation process, the compound bonded to the linking molecule on the substrate in the solid carrier will not be fallen off. That is, the amide bond (or urea functional group) formed between the linking molecule on the substrate and the compound is not broken, and the compound remains on the substrate. Then, the solid carrier bonded with the degraded compound is re-bonded to a protecting group to obtain the solid carrier bonded with the compound represented by Formula (1). Thereby, the solid carrier may be reused.

Hereinafter, the disclosure will be described in detail with embodiments. The following examples are provided to describe the disclosure, and the scope of the disclosure includes the categories described in the following patent claims and their substitutions and modifications, and are not limited to the scope of the examples.

Synthesis Example 1 to Synthesis Example 5, Example 1 to Example 12, Comparative Example 1, Preparation Example 1 to Preparation Example 3 and Preparation Comparative Example 1 are described below.

### Synthesis Example 1: Preparation of Compound 1

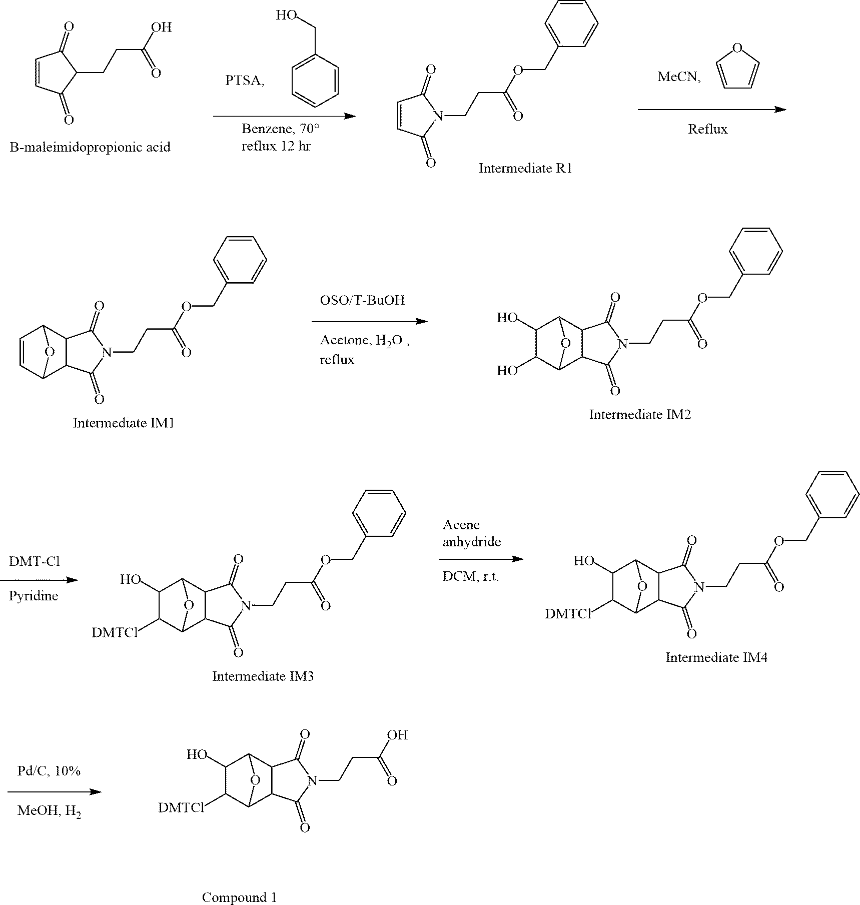

The synthesis was started with 31.3 mmol of ß-maleimidopropionic acid, and after adding 31.3 mmol of para-toluene sulfonic acid (PTSA) thereto, 12.5 mL of benzyl alcohol and 6.5 mL of benzene were added thereto in sequence. After shaking with an ultrasonic oscillator until there are no solid particles in the bottle, a condenser tube was set up and started stirring, and a reflux reaction was performed at 70° C. to 75° C. for 12 hours. After the reaction, dichloromethane (DCM)/pure water was used for extraction, and magnesium sulfate (MgSO₄) was used to remove water, then filtered and concentrated to remove the solvent, and purified by column chromatography to obtain Intermediate R1 (yield: 40% to 57%).

Next, 17.74 mmol of Intermediate R1, acetonitrile (MeCN) and 40.8 mmol of furan were heated and refluxed at 80° C. for 5 hours. After the reaction, the solvent in the reaction solution was directly removed by a vacuum concentration device, and purified by column chromatography to obtain Intermediate IM1 (yield: 59% to 67%).

Then, 4.14 mmol of Intermediate IM1, 10 mL of acetone, and 2.2 mL of hydrogen peroxide (H₂O₂) were stirred to dissolve to form a reaction solution. Separately, 0.0414 mmol of osmium tetroxide (OsO₄) solid was dissolved in 1.1 mL of tertiary butanol (t-BuOH) to form a tertiary butanol solution. Next, the tertiary butanol solution was added dropwise to the reaction solution, and after the tertiary butanol solution was dropped, the temperature was raised to 55° C. to 60° C. to perform reflux reaction for 4 hours. After the reaction was completed, the temperature of the reaction solution was returned to room temperature, and then moved to an ice bath, and a saturated sodium thiosulfate solution was added dropwise to the reaction solution to neutralize the reaction solution. After the neutralization was completed, the reaction solution was in a layered state. At this time, the supernatant was taken and the solvent was removed with a vacuum concentration device, and purified by column chromatography to obtain the Intermediate IM2 (yield: 49 % to 50%).

Next, 3.02 mmol of dimethoxytrityl chloride (4,4′-dimethoxytrityl chloride, DMT-Cl) was pre-dissolved in 4.5 mL of pyridine for which the water had been completely removed to form a solution A. Then, 2.02 mmol of Intermediate IM2 was dissolved in 4.5 mL of pyridine for which the water had been completely removed to form solution B. Next, the solution A was withdrawn and added into the reaction flask of the solution B, and the reaction was continued at room temperature for at least 12 hours. After the reaction was completed, an appropriate amount of toluene was added to the reaction flask, and the solvent was removed directly with a vacuum concentration device, and purified by column chromatography to obtain Intermediate IM3 (yield: 49% to 51 %).

Next, 0.75 mmol of Intermediate IM3 was dissolved in 4 mL of dichloromethane (DCM), and then 0.075 mmol of 4-dimethylaminopyridine (DMAP), 1.13 mmol of triethylamine (TEA) and 1.13 mmol of acetic anhydride were added sequentially to the reaction solution in which the Intermediate IM3 was dissolved, and the reaction was continued at room temperature for 2 hours. After the reaction was completed, a saturated aqueous sodium bicarbonate solution was added dropwise to the reaction solution for neutralization until the pH returned to neutral. After neutralization, the reaction solution was transferred to an extraction bottle, and dichloromethane (DCM) was added in the same amount as the reaction solution. Next, after washing twice with pure water, the organic layer was taken out, filtered and concentrated to remove the solvent after dewatering with magnesium sulfate (MgSO₄), and purified by column chromatography to obtain the Intermediate IM4 (yield: 83% to 93%).

Next, 0.135 mmol of Intermediate IM4 was placed in the reaction flask, and 3 mL of methanol (MeOH), 10 mL of 10% Pd/C powder and 0.135 mmol of triethylamine (TEA) were added thereto sequentially. Then, after replacing the gas with hydrogen (H₂) three times, the reaction was performed at room temperature for 2 hours. After the reaction was completed, the Pd/C residual powder was filtered with sea sand, and the filtrate was collected by filtration to remove the solvent in the filtrate with a vacuum concentration device, and purified by column chromatography to obtain the final product Compound 1 (yield: 91% to 96%).

### Synthesis Example 2: Preparation of Compound 2

The Compound 2 of Synthesis Example 2 were prepared using the same steps, ratio of the amount of reactants and reaction conditions as Synthesis Example 1, and the difference thereof is: the starting compound was changed to ß-maleimidoheptanoic acid. During the preparation of Compound 2, the yields of five intermediates produced sequentially were 68.5%, 79.5%, 65.5%, 28.5% and 85.0%, respectively. The yield of Compound 2 was 90.0%.

### Synthesis Example 3: Preparation of Compound 4

The Compound 4 of Synthesis Example 3 were prepared using the same steps, ratio of the amount of reactants and reaction conditions as Synthesis Example 1, and the difference thereof is: the starting compound was changed to ß-maleimidohexanoic acid. During the preparation of Compound 4, the yields of five intermediates produced sequentially were 51.8%, 99.0%, 31.3%, 49.7% and 89.1%, respectively. The yield of Compound 4 was 95.3%.

### Synthesis Example 4: Preparation of Compound 5

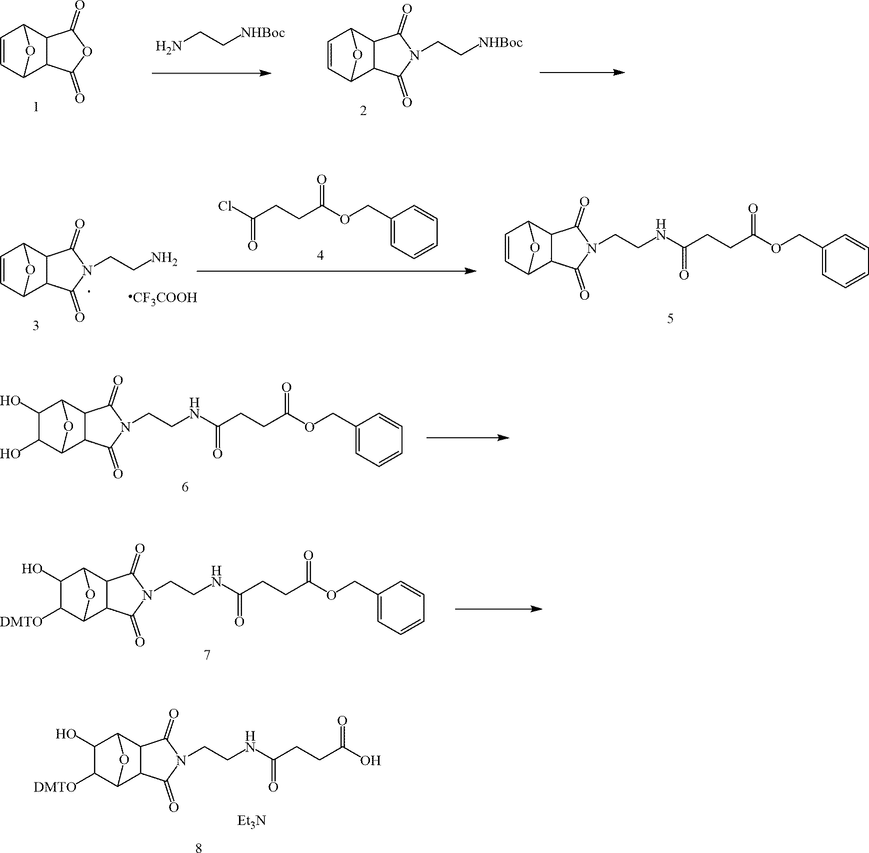

3.93 g of 7-Oxabicyclo[2.2.1]hept-5-ene-2,3-exo,exo-dicarboxylic anhydride (CAS number: 6118-51-0) was dissolved in 30 ml of acetone and stirred. Separately, 3.78 g of tert-butyl 2-aminoethylcarbamate was added to the stirred acetone solution. After about 2 hours, the solution was filtered to obtain a white solid therein, which was washed with acetone and dried to obtain about 6.65 g of a semi-finished product. Next, the semi-finished product, 20 mL of acetone, 5.19 g of acetic anhydride (Ac₂O) and 0.1 g of sodium acetate (CH₃COONa) were mixed, and after heated to reflux for 2 hours, the acetone was removed under reduced pressure, and the remaining residue was added to 10 mL of sodium carbonate saturated aqueous solution (Na₂CO₃ (aq)). Then, after the solid therein was filtered and washed with water, it was taken out and dried to obtain about 2.72 g of Intermediate 2 (yield: 37.3%).

Next, 1.55 g of Intermediate 2 was dissolved in 5 mL of dichloromethane (CH₂Cl₂), and 3 mL of trifluoroacetic acid (CF₃COOH) was added at 0° C., stirred and gradually returned to room temperature, and stirred for 2 hours. After the reaction was completed, the solvent was removed under reduced pressure, and toluene was added, and the toluene was distilled off under reduced pressure and retained to obtain the first batch of residues. Separately, 1.052 g of 4-(benzyloxy)-4-oxobutanoic acid was dissolved in 5 mL of dichloromethane at 0° C. and stirred, and at this time 1.2 g of oxalyl chloride was added, and then carefully added 1 drop of dimethylformamide (DMF), gradually returned to room temperature, and stirred for 1 hour. Next, the solvent was removed from the reaction solution under reduced pressure, and toluene was added again. After evaporation to dryness again, fresh dichloromethane was added to obtain a second batch of solution. The first batch of residues was added to 5 mL of dichloromethane, kept at 0° C. with stirring, and 3 equivalents (eq) of triethylamine (Et₃N) was added carefully. After that, the second batch of dichloromethane solution was added slowly, and then gradually warmed up and stirred for 1 hour. Then, water was added for extraction, and the organic layer was taken out, and washed with 1 N hydrochloric acid (HCl), and then washed with water until neutral. The organic layer was dried and concentrated, and the solvent was removed under reduced pressure to obtain a residue. The residue was added to 2.5 mL of methanol. After standing overnight, the precipitated solid was filtered and washed with ice methanol, then the solid was taken out and dried to obtain about 0.768 g of Intermediate 5 (yield: 38.3%).

Next, 0.644 g of Intermediate 5 was dissolved in 7 mL of acetone, and then 2.3 mL of hydrogen peroxide was added for stirring. In addition, after dissolving 3.4 mmol of osmium tetroxide (OsO₄) in 1 mL of tert-butanol, it was added dropwise to the aforementioned acetone solution, and stirred at room temperature. After stirring overnight, a solid precipitated out. The solid was firstly filtered, then most of the acetone was removed from the filtrate under reduced pressure, then sodium thiosulfate aqueous solution (Na₂S₂O₃ (aq)) was added, and stirred slowly, and it may be observed that the white substance was slowly formed. After about 2 hours, the solid was filtered and washed with 3 mL of dichloromethane. The solid was dried to obtain about 0.197 g of Intermediate 6 (yield: 28%).

Next, another reaction bottle was taken. 3 mL of dichloromethane was firstly added thereto, then 0.2821 g of Intermediate 6 and 0.331 g (1.5 equivalents) of dimethoxytrityl chloride (DMT-Cl) were added, and finally 2.5 equivalents of triethylamine and 5 mol of 4-dimethylaminopyridine (DMAP) were slowly added. After the reaction solution returned to room temperature, it was stirred at room temperature for 3.5 hours, and then water was added and stirred to separate layers. After the organic layer was taken out and dried, the solvent was removed under reduced pressure and a residue was obtained. The residue was purified by column chromatography to obtain about 0.217 g of Intermediate 7 as an oil (yield: 45%).

Next, 20 mg of Intermediate 7 was dissolved in 3 mL of methanol, and then 3 equivalents of triethylamine and 7 mg of 10% Pd/C were added thereto, and reacted for 1.5 hours under a hydrogen pressure of 1.5 atm. Then, the reaction solution was filtered to remove the Pd catalyst. Next, the solvent in the reaction solution was removed to obtain the final solid product Compound 5 (yield: 92%).

### Synthesis Example 5: Preparation of Compound 6

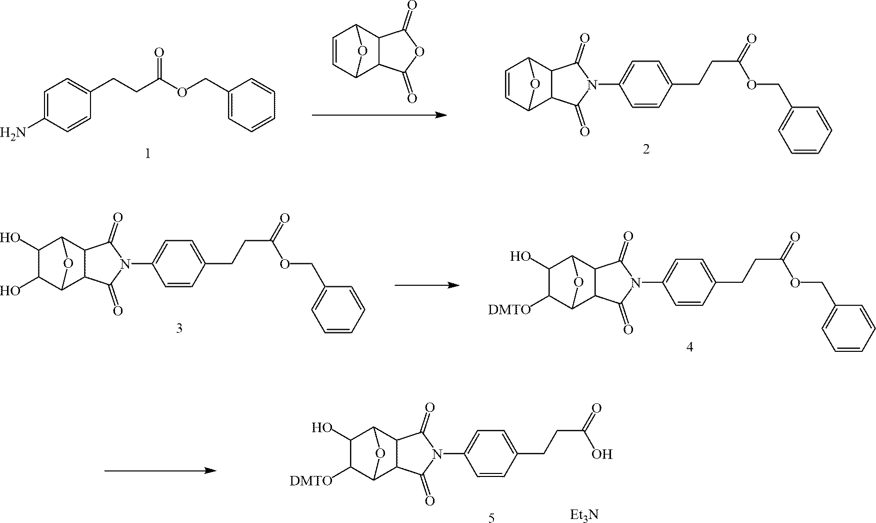

2.687 g of benzyl 3-(4-aminophenyl)propanoate was weighed and dissolved in 15 mL of acetone and stirred. Separately, 1.93 g of 7-Oxabicyclo[2.2.1]hept-5-ene-2,3-exo,exo-dicarboxylic anhydride were added to the stirred acetone solution. After about 2 hours, the solution was analyzed by thin layer chromatography (TLC) to confirm whether the reaction is complete. After confirming that the reaction was completed, 2.5 equivalents of acetic anhydride (Ac₂O) and 0.5 equivalents of sodium acetate (CH₃COONa) were added, and after heated to reflux for 3 hours, acetone was removed under reduced pressure, and the remaining residue was added to 10 mL of saturated aqueous solution of sodium carbonate (Na₂CO₃ (aq)). Then, after stirring for 30 minutes, ethyl acetate (EA) was added for extraction. Next, the extract was dried and concentrated, purified by column chromatography, and crystallized again in methanol to obtain about 1.9 g of Intermediate 2 (yield: 44.7%).

25.4 mg of Intermediate 2 was dissolved in 2 mL of acetone, and then 0.2 mL of hydrogen peroxide was added for stirring. In addition, after dissolving 1.8 mg of osmium tetroxide (OsO₄) in 0.2 mL of tert-butanol, it was added dropwise to the aforementioned acetone solution, and stirred. After stirring overnight, a large amount of water was added to precipitate a white solid. The solid was filtered, washed with water, and dried to obtain about 17 mg of Intermediate 3 (yield: 61%).

Next, another reaction bottle was taken. 3 mL of dichloromethane was firstly added thereto, then 0.167 g of Intermediate 3 and 0.081 g (1.3 equivalents) of dimethoxytrityl chloride (DMT-Cl) were added, and finally 2.5 equivalents of triethylamine and 5 mol of 4-dimethylaminopyridine (DMAP) were slowly added. After the reaction solution was stirred overnight at room temperature, water was added and stirred to separate layers. Then, after the organic layer was taken out and dried, a reduced pressure was performed to remove the solvent, and a residue was obtained. The residue was added back into ethyl acetate to filter the insoluble matter, and the organic layer containing ethyl acetate was taken back up to remove the solvent. Then, purification was performed by column chromatography to obtain an oil. Next, methanol was added to precipitate solid Intermediate 4 (yield: 40% to 50%).

Next, 22 mg of Intermediate 4 was dissolved in 3 mL of methanol, and then 3 equivalents of triethylamine and 8 mg of 10% Pd/C were added, and reacted for 2 hours under a hydrogen pressure of 1.5 atm. Then, the reaction solution was filtered to remove the Pd catalyst. Next, the solvent in the reaction solution was removed to obtain the final solid product Compound 6 (yield: 95%).

### Example 1

0.05 mol of aminopropyltriethoxysilane was dissolved in methanol, and then added to column A and let it stay for 24 hours. Next, the column was washed with acetone to remove methanol, and dried at a temperature of 60° C. Then, 0.05 mol of Compound 1 was dissolved in dichloromethane, and then added to column A for 24 hours. Next, the column was washed with acetone to remove dichloromethane, and dried at a temperature of 60° C. to obtain the solid carrier of Example 1. The obtained solid carrier was evaluated by the following evaluation methods, and the results thereof are shown in Table 2 and Table 3.

### Example 2 to Example 12

The solid carriers of Example 2 to Example 12 were prepared using the same steps as Example 1, and the difference thereof is: the compound bonded to the substrate and the column were changed (as shown in Table 2), wherein the compounds/columns corresponding to the symbols in Table 2 are shown in Table 1. The obtained solid carriers were evaluated by the following evaluation methods, and the results thereof are as shown in Table 2 and Table 3.

### Comparative Example 1

Commercially available product DS200 (trade name, manufactured by K&A Labs GmbH) was used, which includes a compound that may bind to a nucleic acid precursor. The DS200 (trade name) was evaluated by the following evaluation methods, and the results thereof are as shown in Table 2 and Table 3.

### Preparation Example 1 to Preparation Example 3 and Preparation Comparative Example 1 of Nucleic Acid

The solid carriers obtained in Example 3 to Example 5 and the solid carrier of Comparative Example 1 were respectively loaded into a K&A synthesizer (manufactured by K&A Labs GmbH) for preparing 5′ TGA CTG TGA ACG TTC GAG ATG A sequence nucleic acid. The columns that completed preparing the nucleic acid were evaluated by the following evaluation methods, and the results are shown in Table 4.

TABLE 1

| | | |
|---|---|---|
| | Symbol | Components/compound |
| Compound | Compound 1 | Compound represented by Formula (1-1) |
| | Compound 2 | Compound represented by Formula (1-2) |
| | Compound 4 | Compound represented by Formula (1-3) |
| | Compound 5 | Compound represented by Formula (1-4) |
| | Compound 6 | Compound represented by Formula (1-5) |
| Column | Column A | V2-A1 (Self-made column, wherein the linking molecule on the surface is aminopropyltriethoxysilane). |
| | Column B | V5-B11 (Self-made column, wherein the linking molecule on the surface is aminopropyltriethoxysilane). |
| | Column C | V6-C16 (Self-made column, wherein the linking molecule on the surface is aminopropyltriethoxysilane). |
| | Column D | DS200 (trade name, manufactured by K&A Labs GmbH). |

TABLE 2

| Component | | Examples | | | | | |
|---|---|---|---|---|---|---|---|
| | | 1 | 2 | 3 | 4 | 5 | 6 |
| Compound | Compound 1 | V | V | - | - | - | - |
| | Compound 2 | - | - | V | - | - | - |
| | Compound 4 | - | - | - | V | - | - |
| | Compound 5 | - | - | - | - | V | |
| | Compound 6 | - | - | - | - | - | V |
| Column | Column A | V | - | - | - | - | |
| | Column B | - | V | V | V | V | V |
| | Column C | - | - | - | - | - | - |
| | Column D | - | - | - | - | - | - |
| Number of binding sites (Unit: nmol) | | 447 | 331 | 228 | 280 | 287 | 194 |

TABLE 2 (Continued)

| Component | | Examples | | | | | | Comparative Example |
|---|---|---|---|---|---|---|---|---|
| | | 7 | 8 | 9 | 10 | 11 | 12 | 1 |
| Compound | Compound 1 | V | - | - | - | - | - | - |
| | Compound 2 | - | V | - | - | - | - | - |
| | Compound 4 | - | - | V | | | V | - |
| | Compound 5 | - | - | - | V | - | - | - |
| | Compound 6 | - | - | - | - | V | - | - |
| Column | Column A | - | - | - | - | - | V | - |
| | Column B | - | - | - | - | - | - | - |
| | Column C | V | V | V | V | V | - | - |
| | Column D | - | - | - | - | - | - | V |
| Number of binding sites (Unit: nmol) | | 582 | 433 | 460 | 478 | 319 | 475 | 200 |

TABLE 3

| | Examples | | Comparative Example |
|---|---|---|---|
| | 1 | 12 | 1 |
| Recovery number (Unit: nmol) | 326 | 296 | 0 |
| Recovery ratio (Unit: %) | 73 | 62 | 0 |

TABLE 4

| | Preparation Examples | | | Preparation Comparative Example |
|---|---|---|---|---|
| | 1 | 2 | 3 | 1 |
| Solid carrier | Example 3 | Example 4 | Example 5 | Comparative Example 1 |
| Crude yield (Unit: ng/µL) | 131 | 70 | 107 | 185 |

### Evaluation Methods

### A. Number of Binding Sites

The prepared solid carrier was soaked with 3% trichloroacetic acid (TCA) for 1 minute, so that the trichloroacetic acid degraded the dimethoxytrityl group on the compound and exposed a site (i.e., a binding site) which may bind with the nucleic acid precursor. Next, the concentration of dimethoxytrityl group in the solution was measured to estimate the equivalent number of binding sites.

### B. Recovery Number, Recovery Ratio

The aforementioned solid carrier with the measured dimethoxytrityl concentration (that is, the solid carrier bonded with the degraded compound) was put in a solution formed by dimethoxytrityl chloride and pyridine (DMT-Cl/Pyridine) to allow the compound to be rebonded to the protecting group. After 24 hours, it was soaked with 3% trichloroacetic acid (TCA) for 1 minute, so that the trichloroacetic acid degraded the dimethoxytrityl group on the compound and exposed a site (i.e., a binding site) which may bind with the nucleic acid precursor. Next, the concentration of dimethoxytrityl group in the solution was measured to estimate the equivalent number of binding sites to obtain a recovery number. And a recovery ratio was obtained by calculating the recovery number and the number of binding sites.

### C. Crude Yield

The sample of nucleic acid was analyzed via an ultraviolet light absorption instrument: concentration value (unit: ng/µL) = (A₂₆₀ reading value - A₃₂₀ reading value) × dilution ratio × 33 ng/µL (concentration determination of single-stranded nucleic acid) × sample volume. The crude yield was obtained.

### Evaluation Results

As may be seen from Table 2 and Table 3, the solid carriers prepared by the compound having specific structure (Examples 1 to 12) have good number of binding sites, recovery number and recovery ratio, and may be reused and suitable for preparing a nucleic acid. On the other hand, the number of binding sites of the solid carrier for which the compound does not include specific structure (Comparative example 1) is not good, and the solid carrier cannot be recycled, that is, cannot by reused.

In addition, in the case of using the same column, compared to the solid carriers prepared by Compound 2 or Compound 6 (Examples 3, 6, 8, 11), the solid carriers prepared by Compound 1, Compound 4 or Compound 5 (Examples 1, 2, 4, 5, 7, 9, 10, 12) have more number of binding sites, that is, better number of binding sites. Therefore, when the compound has a structure represented by Formula (1) and Z is alkylene group, *—COO—* or a combination thereof, the solid carrier prepared by the compound may have better number of binding sites; specifically, the alkylene group is an alkylene group having 2 to 5 carbon atoms. When the compound has a structure represented by Formula (2) and Z¹ is *—COO—*, m is an integer from 2 to 5 and n is 0, the solid carrier prepared by the compound may have better number of binding sites.

In addition, as may be seen from Table 4, when the solid carrier prepared by the compound having specific structure is used to prepare nucleic acid (Preparation example 1 to 3), a good crude yield of nucleic acid is obtained. As may be seen from Table 3 and Table 4, compared to the solid carrier prepared by the compound for which the compound does not have specific structure used to prepare nucleic acid (Preparation comparative example 1), the solid carrier prepared by the compound having specific structure used to prepare nucleic acid has good crude yield of nucleic acid and the solid carrier can be recycled for reuse.

Based on the above, the exemplary embodiments provide binding a compound having a structure represented by Formula (1) on the surface of the substrate to let the compound form an amide bond or a urea functional group with the linking molecule on the surface of the substrate, so that the solid carrier including the compound having a specific structure has better number of binding sites, and is suitable for preparing a nucleic acid. In addition, the solid carrier including the compound having a specific structure can be recovered and reused after the nucleic acid is prepared. Thereby, the solid carrier has reusable property, which may contribute to reducing the cost of preparing a nucleic acid.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A compound, having a structure represented by Formula (1) as follows: 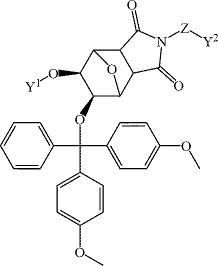
in Formula (1), Y¹ represents hydrogen or acetyl group;
Y² represents hydrogen, *—COOH, *—NH₂ or *—NCO;
Z represents a divalent organic group, and
* represents a bonding position.

2. The compound of claim 1, wherein Z comprises alkylene group, *—COO—*, arylene group, *—HNCO—*, *—NHCONH—*, heterocycloalkylene group, heteroarylene group or a combination thereof, * represents a bonding position.

3. The compound of claim 1, wherein the structure represented by Formula (1) comprises a structure represented by Formula (2) as follows: 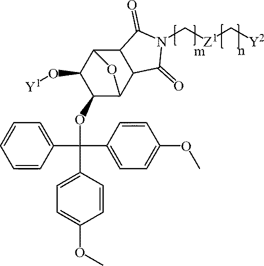
in Formula (2), Y¹ represents hydrogen or acetyl group;
Y² represents hydrogen, *—COOH, *—NH₂ or *—NCO;
Z¹ represents a divalent organic group;
m represents an integer from 0 to 8;
n represents an integer from 0 to 6, and
* represents a bonding position.

4. The compound of claim 3, wherein Z¹ comprises *—COO—*, arylene group, *—HNCO—*, *—NHCONH—*, heterocycloalkylene group, heteroarylene group or a combination thereof, * represents a bonding position.

5. A solid carrier, comprising the compound of claim 1.

6. The solid carrier of claim 5, further comprising a substrate whose surface has a linking molecule, the compound bonding to the linking molecule.

7. The solid carrier of claim 6, wherein a terminal functional group of the linking molecule comprises amino group or carboxyl group, and the terminal functional group bonds to the compound.

8. The solid carrier of claim 6, wherein an amide bond or a urea functional group is formed between the compound and the linking molecule.

9. The solid carrier of claim 7, wherein an amide bond or a urea functional group is formed between the compound and the linking molecule.

10. A method for preparing a nucleic acid, comprising using the solid carrier of claim 5.

11. The method for preparing a nucleic acid of claim 10, wherein the solid carrier is a reusable solid carrier.
